(19) United States
(12) Patent Application Publication
(10) Pub. No.: US 2020/0321107 A1
(43) Pub. Date: Oct. 8, 2020

(54) INTEGRATED MULTI-FACILITY ELECTRONIC MEDICAL RECORD SYSTEM

(71) Applicant: IRYOU JYOUHOU GIJYUTU KENKYUSYO CORPORATION, Yame-gun Fukuoka (JP)

(72) Inventor: Shinkichi HIMENO, Yame-gun Fukuoka (JP)

(21) Appl. No.: 16/884,584

(22) Filed: May 27, 2020

Related U.S. Application Data
(63) Continuation-in-part of application No. 15/574,694, filed on Nov. 16, 2017.

(30) Foreign Application Priority Data
May 19, 2015 (JP) 2015-101506

(51) Int. Cl. G16H 40/20; G16H 10/60; G16H 50/70; G06Q 10/10; H04L 29/06; G06F 21/62; G06F 16/93; G06F 16/906

(52) U.S. Cl. CPC G16H 40/20; G16H 10/60; G16H 50/70; G06Q 10/10; G06F 2221/2141; H04L 63/101; G06F 21/6209; G06F 16/93; G06F 16/906; H04L 63/083

(57) ABSTRACT

To achieve smoothly coordinated activities among medical facilities, care facilities, and welfare facilities by enabling creating and referencing of consistent records and work instruction documents among the facilities even in a case where a patient is transferred from a hospital to a care facility to a home care service.

An integrated multi-facility electronic medical record system includes: a facility-specific document category managing unit that manages document categories being used at facilities; a facility-specific document-category-specific document recording unit that records document data for each document category at each facility; and a facility-specific document-category-specific access right managing unit that manages access rights of staff for each document category at each facility. The facility-specific document-category-specific access right managing unit includes a staff login authenticating unit that authenticates staff for each document category at each facility, and the integrated multi-facility electronic medical record system further includes: an accessible document referencing unit that references, for each facility and each document category, a document the staff have rights to access, the document relating to a designated user; and a multi-facility document display unit that displays a referenced multi-facility document.

### TECHNICAL FIELD

The present invention relates to an electronic medical record system that efficiently creates and references records and work instruction documents on patients and residents at medical facilities, care facilities, and welfare facilities. More particularly, the present invention relates to an integrated multi-facility electronic medical record system that achieves smoothly coordinated activities among medical facilities, care facilities, and welfare facilities by enabling creating and referencing of consistent records and work instruction documents among the facilities even in a case where document category combinations vary with facilities, where staff members in a wide variety of jobs intermix in a complicated manner between the facilities, or where a patient is transferred from a hospital to a care facility to a home care service.

### BACKGROUND ART

As the number of elderly people in need of nursing care due to chronic disease or dementia has been increasing in recent years, it is becoming difficult to complete medical and nursing care only at a hospital or a care facility. A patient who has received treatment at a hospital due to pneumonia or a broken bone or the like is often unable to return to his/her home immediately after hospital discharge. In many cases, such a patient enters a care facility in the community, and, after the symptoms are alleviated, returns to his/her home or a home care facility while receiving home examination and home treatment and care provided by staff from a clinic in the community. In this manner, a patient is transferred from a hospital or a clinic to a care facility and then to a home care facility or his/her home. Along with the transfer, people in a wide variety of jobs, such as doctors, nurses, rehabilitators, caregivers, and pharmacists, are involved in the medical and nursing care. The need to establish a so-called “community comprehensive care system”, in which detailed information is shared among facilities and jobs, and organic, cooperative operations are performed, is now being suggested. In such a trend, it is inevitable to form an integrated multi-facility electronic medical record system that enables sharing of detailed information between facilities and jobs, and organic, cooperative operations.

In a conventional medical information sharing system of this kind, respective hospitals are connected by a network, and patient information gathered in a server is shared and viewed in the system. In a medical information disclosure system of this kind, the following technology is adopted, for example: electronic medical record information from hospitals A through C and the like is accumulated in a shared server, so that a hospital D, clinics E and F, and the like can access the shared server through Internet browsers, and reference necessary medical treatment information, as shown in FIG. 1.

In a case where the clinic E outside the group uses the system, the clinic E obtains a right to use an ID, a password, and the like, and logs in the central server via a network.

As a technology of this kind, the technology disclosed in Patent Literature 1 has been suggested.

### CITATION LIST

### Patent Literature

### Patent Literature 1: JP 2002-117142 A

In recent years, the mode shown in FIG. 2 has been becoming popular. In this mode, a central ID management server manages only the ID information about the target patients, and does not hold any medical treatment information at all. The ID management server that manages patient IDs is installed at the center of a network. Hospitals A through C that provide medical treatment information are connected to the system via a gateway server unit (GSu). Only the ID information about patients who have consented to information disclosure from their facility is transmitted from each medical treatment facility to the ID management server. In a case where a target patient has received treatment from more than one medical organization, the patient IDs at the respective medical organizations are transmitted to the ID management server. The ID management server issues a unique ID management number, to associate the patient IDs at the respective medical organizations with one another.

For example, a clinic D that wishes to reference the medical treatment information about a patient sends an inquiry to the ID management server about the IDs of the patient, and then receives the patient IDs of the patient at the respective hospitals A through C that hold medical treatment information about the patient, and a right to access the medical treatment information at these hospitals. Using these IDs, the clinic D sends a medical treatment information transmission request to the gateway server units of the medical organizations, and obtains the display shown in FIG. 3 with the transmitted medical treatment information.

### SUMMARY OF INVENTION

### Technical Problem

In Conventional Example 1 shown in FIG. 1, medical treatment data to be disclosed needs to be recorded in the central shared server. Therefore, an enormous storage capacity is required, and the costs for constructing and operating the system are high. Further, once unauthorized access is made, there is a risk of leakage of a large amount of medical treatment information. Furthermore, since the combinations of document categories in electronic medical records and the particulars in the respective documents vary with facilities, it is not possible to simultaneously view the related electronic medical record data of different facilities in chronological order, though it is possible to view the electronic medical records of each individual facility.

Unlike the system of Conventional Example 1, the system of Conventional Example 2 shown in FIG. 2 manages only patient IDs, and requires a small-sized server accordingly. Thus, even if unauthorized person accesses the server, the danger of data theft and loss can be small. Also, it is possible to reference the data of the medical organizations in the network in chronological order. In the network system shown in FIG. 2, however, the management form is complicated, and moreover, there are the problems described below.

(1) The two products, ID-link and HumanBridge, are available at the present time. However, the browser display implementation methods completely differ between the two products. Therefore, medical treatment data provided by hospitals using different products from each other cannot be simultaneously displayed on the same browser screen. Because of this, not all the medical treatment data can be displayed in the same chronological sequence. Therefore, accurate lists cannot be formed, and the flow of medical treatment is not easily recognized. As a result, a medical accident might occur due to overlooking of medical treatment information.

(2) Each of the products needs to collectively convert the document categories and the particulars that differ with facilities into those compliant with a unified standard called SS-MIX. This conversion process requires some time and costs, and therefore, only part of electronic medical record information can be shared at the present time. Furthermore, standards for care facilities, home visit services, and nursing care have not yet been established, and it is difficult to share information necessary for comprehensive care in the community after hospital discharge.

(3) Each of the products is in such a mode to unidirectionally provide hospitals' electronic medical record data to clinics and the like in the community, and therefore, bidirectional information exchange between staff members with different job titles at hospitals, clinics, care facilities, and home visit services necessary for comprehensive care in the community is difficult in practice. Further, each of the products is basically designed to only reference documents, and the system is not configured to create and edit instruction documents and records about any patient at any particular facility.

(4) In comprehensive care in the community, home-visit nursing staff and home-visit care staff conduct work at facilities and patients' homes. For example, a certified bedsore care nurse takes care of inpatients and outpatients at the hospital to which he/she belongs. Such a certified bedsore care nurse also belongs to a home-visit nurse station, and visits care facilities and patients' homes to take care of patients. In this manner, a staff member belongs to more than one organization, and provides services at more than one facility. In such complicated working arrangements, it is necessary to appropriately control the rights to create and view documents. However, each of Conventional Examples 1 and 2 is based on the assumption that each one medical staff member belongs to only one facility, and is not based on the above described complicated right control operation in which facilities as the sites to provide services do not match the staff organizations to provide services.

As the services to be provided vary with facilities, the document categories being used and the particulars in the respective documents slightly differ among the facilities. In conventional cases, even document categories with similar contents are displayed as completely different document categories, unless the document categories have exactly the same names and exactly the same particulars. Therefore, it is difficult to collectively view the contents in chronological order. Meanwhile, converting document categories into unified document categories such as those in SS-MIX requires a lot of time and costs, and therefore, it is not realistic to convert all the document categories at all the facilities. In view of this, in conventional examples, it is difficult to view medical and nursing care information, in chronological order, about patients being transferred between facilities, while appropriately managing the rights of staff members working at different facilities among which document categories and particulars vary. Also, each of the conventional examples is basically designed to only reference documents, and the system is not configured to create and edit instruction documents and records about any patient at any particular facility.

The present invention has been made to solve the above problems of conventional technologies, and an object of the present invention is to provide an integrated multi-facility electronic medical record system that achieves smoothly coordinated activities among medical facilities, care facilities, and welfare facilities by enabling referencing, creating, editing, and deleting of consistent records and work instruction documents among the facilities even in a case where document category combinations vary with facilities, where staff members in a wide variety of jobs intermix in a complicated manner between the facilities, or where a patient is transferred from a hospital to a care facility to a home care service.

### Solution to Problem

As a means to achieve the above object, an integrated multi-facility electronic medical record system of claim 1 characteristically includes: a facility-specific user ID managing unit that manages IDs of facility users at each facility among a plurality of facilities; a facility-specific document category managing unit that manages document categories being used at the facilities; a facility-specific document-category-specific document recording unit that records document data for each document category at each facility; and a facility-specific document-category-specific access right managing unit that manages access rights of staff for each document category at each facility,

the facility-specific document-category-specific access right managing unit including a staff login authenticating unit that authenticates staff for each document category at each facility,

the integrated multi-facility electronic medical record system further including: an accessible document referencing unit that references, for each facility and each document category, a document the staff have rights to access at the facilities, the document relating to a designated user; and a multi-facility document display unit that displays a referenced multi-facility document on the terminal based on the referenced document data with the tags transmitted to the terminal.

An integrated multi-facility electronic medical record system of claim 2 is an integrated multi-facility electronic medical record system of claim 1, wherein the multi-facility document display unit includes an inter-facility similar document category associating unit that displays, in similar manners, documents classified under similar document categories at different facilities.

An integrated multi-facility electronic medical record system of claim 3 is the integrated multi-facility electronic medical record system of claim 1 or 2, wherein the accessible document referencing unit includes a reference source tag attaching unit that attaches tags to referenced document data, the tags each indicating a reference source.

An integrated multi-facility electronic medical record system of claim 4 is the integrated multi-facility electronic medical record system of any of claims 1 to 3, wherein the staff login authenticating unit includes a document-edit-allowed facility limiting unit that designates a principal facility, and allows document creation, edit, and deletion only in a document category in which the staff have rights to create, edit, and delete a document among document categories at the designated principal facility.

An integrated multi-facility electronic medical record system of claim 5 is the integrated multi-facility electronic medical record system of claim 4, wherein the document-edit-allowed facility limiting unit includes a user-specific currently-used facility managing unit that records, for each user, a facility being currently used, a facility to be used in the future, and a period of use, and, when a user and a date are designated, automatically designates the principal facility.

### Advantageous Effects of Invention

In the integrated multi-facility electronic medical record system of claim 1, the facility-specific user ID managing unit that manages the IDs of facility users for each facility is provided at a plurality of facilities. Thus, a list of users is created for each facility, and user information can be shared among the facilities in accordance with the list.

As the integrated multi-facility electronic medical record system includes the facility-specific document category managing unit that manages the document categories being used at the facilities, and the facility-specific document-category-specific document recording unit that records document data for each document category at each facility, a document in a designated document category is created or edited in accordance with information about the particulars of each of the document categories. The created or edited document is recorded by the facility-specific document-category-specific recording unit of the document category.

As the integrated multi-facility electronic medical record system includes the facility-specific document-category-specific access right managing unit that manages access rights of staff for each document category at each facility, it is possible to access only an allowed range of document categories.

As the facility-specific document-category-specific access right managing unit includes the staff login authenticating unit that authenticates the staff for each document category at each facility, unauthorized access is rejected.

The integrated multi-facility electronic medical record system includes: the accessible document referencing unit that references, for each facility and each document category, a document the staff have rights to access at the facilities, the document relating to a designated user; and the multi-facility document display unit that displays a referenced multi-facility document. Thus, documents of facilities are displayed as a list.

In the integrated multi-facility electronic medical record system of claim 2, the multi-facility document display unit includes an inter-facility similar document category associating unit that displays, in similar manners, documents classified under similar document categories at different facilities. Even in a case where a document category has different names and particulars depending on facilities, similar documents highly related to one another are displayed in the same category frame. Thus, it becomes easier to understand the contents.

In the integrated multi-facility electronic medical record system of claim 3, the accessible document referencing unit includes a reference source tag attaching unit that attaches tags to referenced document data, the tags each indicating a reference source. Thus, the origins of documents become clear.

Further, it becomes easier to display documents of each facility. When there is a document edit request from a staff member, it becomes clear at which facility an inquiry as to whether the staff member has a right to edit the document should be made.

In the integrated multi-facility electronic medical record system of claim 4, the staff login authenticating unit includes a document-edit-allowed facility limiting unit that designates a principal facility, and allows document creation, edit, and deletion only in a document category the staff have rights to create, edit, and delete among document categories at the designated principal facility. To create, edit, or delete a document in a future principal facility as well as in the current principal facility, the principal facility is designated, and a right to create, edit, or delete a document in the facility is authenticated through the staff login authenticating unit.

In the integrated multi-facility electronic medical record system of claim 5, the document-edit-allowed facility limiting unit includes a user-specific currently-used facility managing unit that records, for each user, a facility being currently used, a facility to be used in the future, and a period of use, and, when a user and a date are designated, automatically designates the principal facility. With this arrangement, a table that manages current and, if necessary, future principal facilities is created for each user, and a user ID and a date on which a document is to be created, edited, or deleted are designated. In this manner, the corresponding principal facility is identified.

### BRIEF DESCRIPTION OF DRAWINGS

FIG. 1 is a diagram for explaining a medical information disclosure network system according to Conventional Example 1.

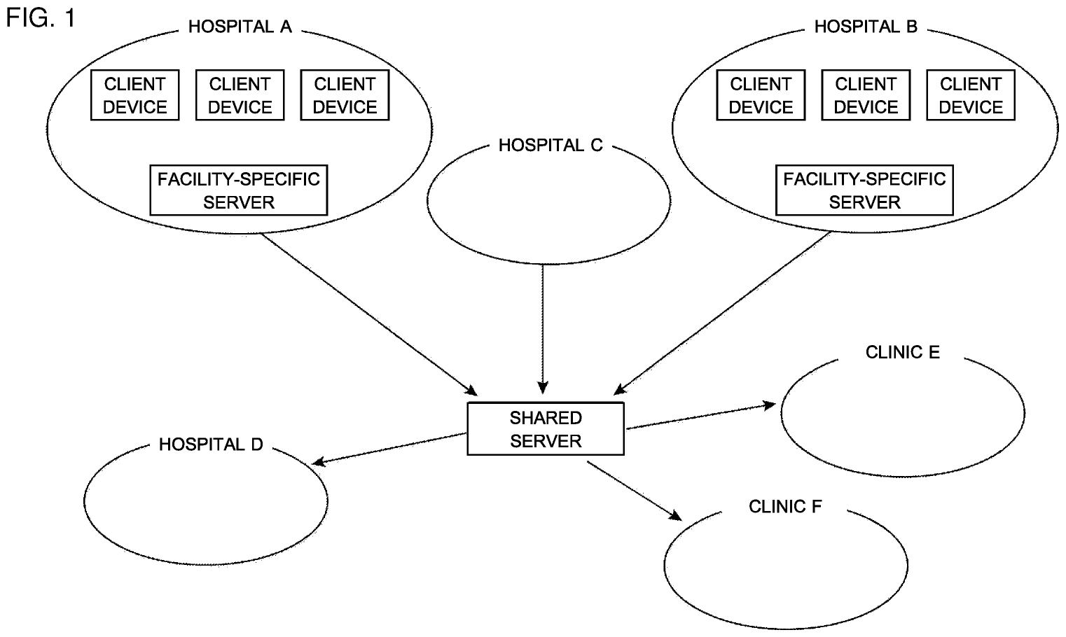

FIG. 2 is a diagram for explaining a medical information disclosure network system according to Conventional Example 2.

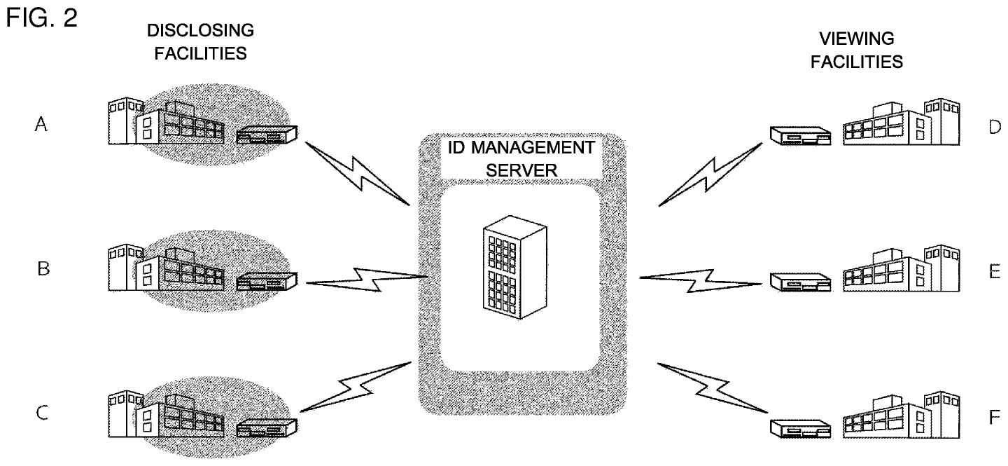

FIG. 3 is an example of a screen display of the medical information disclosure network system according to Conventional Example 2.

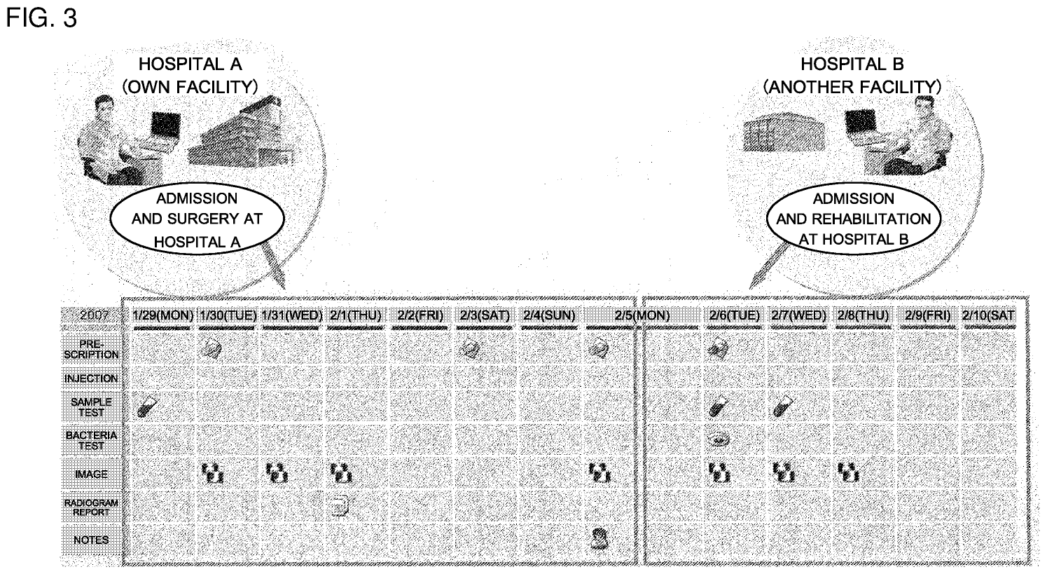

FIG. 4 is a diagram showing the configuration of the inside of a facility in the configuration of an entire integrated multi-facility electronic medical record system of the present invention.

FIG. 5 is a rights management table of the respective staff members in a facility-specific document-category-specific access right managing unit.

FIG. 6 is a diagram showing the configuration of an entire integrated multi-facility electronic medical record system of the present invention.

FIG. 7 is a table in which a multi-facility document display unit of the present invention is used in conjunction with an inter-facility similar document category associating unit.

FIG. 8 is a table showing the doctor-related items of the same contents as those shown in FIG. 7, in chronological order in the respective similar document categories.

### DESCRIPTION OF EMBODIMENTS

### Embodiment

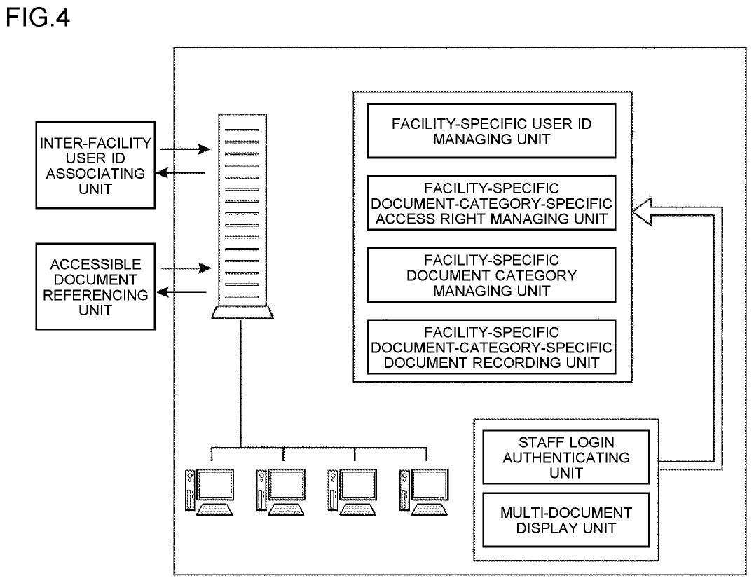

FIG. 4 shows the configuration of the inside of a facility in the configuration of an entire integrated multi-facility electronic medical record system of the present invention. At each facility, there is a facility-specific user ID managing unit that manages the IDs of users who use the facility. The facility-specific user ID managing unit is an inpatient list and an outpatient list at a hospital, is a resident list at a facility, and is a visit destination list or the like in a visit service. For ease of use, each list is in room number order, alphabetical order, ID number order, or the like.

In the facility-specific user ID managing unit, user IDs may be defined at each facility as in a conventional case. In such a case, however, there is a need to create and manage a user ID correspondence table between facilities, to associate and manage the same person with different user IDs between facilities (an inter-facility user ID associating unit). Meanwhile, Medical IDs (provisional name) that are currently considered are unique IDs that can be used throughout the nation, and accordingly, can be shared at all facilities. Thus, the inter-facility user ID associating unit becomes unnecessary, which is the most preferable aspect. The next best measure is to use user IDs that are shared among the respective facilities in the same legal entity. In this case, the above mentioned inter-facility user ID associating unit is used to associate the user IDs with user IDs being used at facilities outside the legal entity. In other words, the inter-facility user ID associating unit associates different user IDs indicating same user between the facilities.

Further, document categories at each facility and particulars (form definitions) of each document category at each facility are determined in accordance with the services being provided at each individual facility. A list of the names of the document categories and information about the particulars of each document category are recorded and managed by the facility-specific document category managing unit. A document in a designated document category is created or edited in accordance with the information about the particulars of the document category. The created or edited document is recorded by a facility-specific document-category-specific recording unit of the document category.

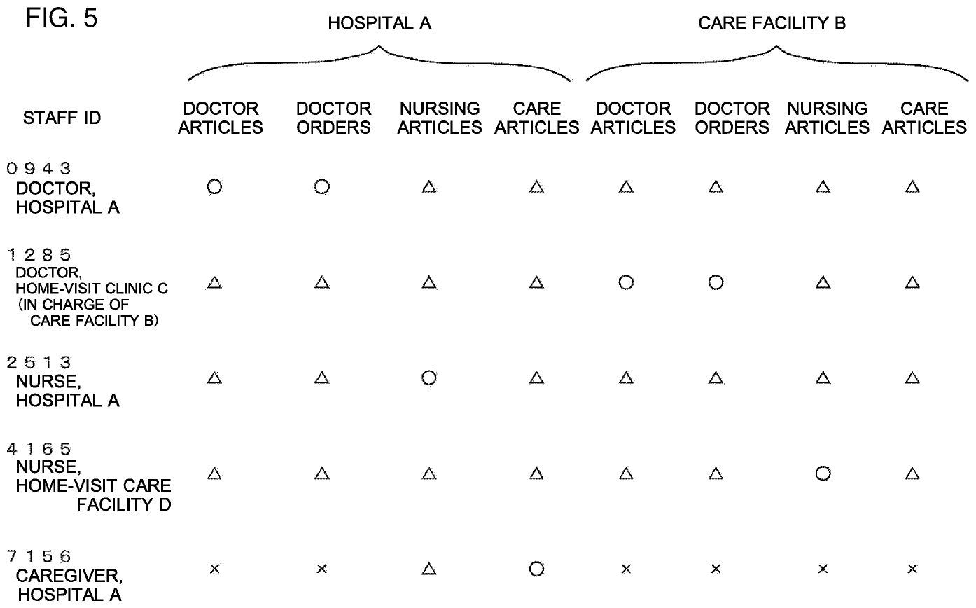

At each facility, combinations of rights of access to the respective document categories, such as document categories that can be created and edited, document categories that can only be referenced, and document categories that are not allowed even to be referenced, vary depending on the affiliation and the types of jobs of the staff members. In the facility-specific document-category-specific access right managing unit, the management table shown in FIG. 5 is created, and access rights are managed for each staff member and each document category. Here, “∘” indicates that creation, edit, and reference are allowed, “Δ” indicates that only reference is allowed, and “x” indicates that even reference is not allowed. For example, a medical doctor who works at the facility can create, edit, and reference doctor-related article documents and documented medical work instructions and prescriptions (order documents), but can only reference articles for the other types of jobs such as nurses and caregivers. As for the doctor articles and the order documents of a facility at which a certain medical doctor is not working, the medical doctor can reference a range of document categories allowed by the facility (or for which an access right is given to the medical doctor), but cannot create or edit any document.

As a staff ID and a password or a biometric identification or the like is input from a terminal, the staff member is authenticated (a staff login authenticating unit). As the staff member is authenticated, the access rights of the staff member in each document category are made clear by the facility-specific document-category-specific access right managing unit. In response to an access request to create, edit, or reference a document category designated by the staff member, the access is allowed if the staff member has the right to do so, but the request is rejected if the staff member does not have the right.

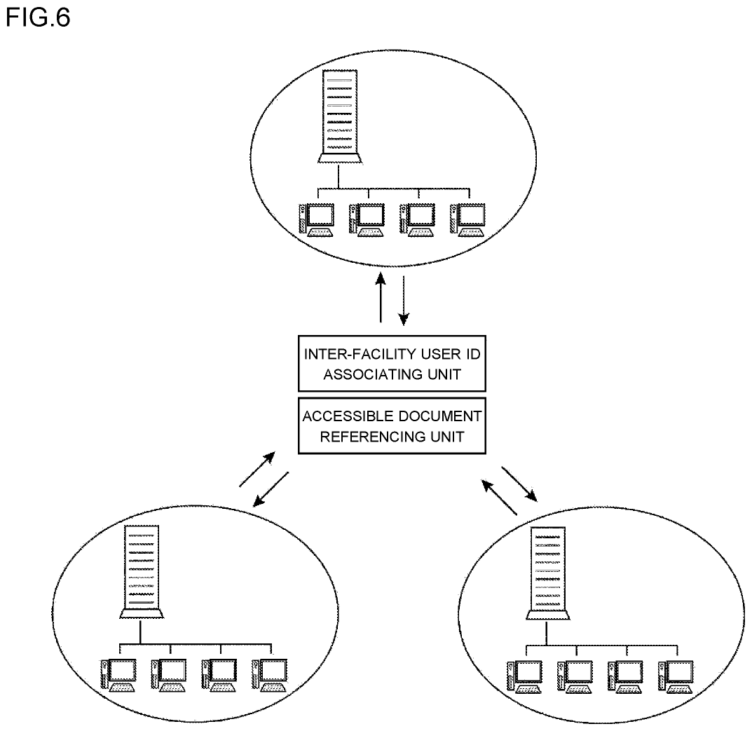

FIG. 6 shows the configuration of the entire integrated multi-facility electronic medical record system of the present invention. In a case where a user receives services at more than one facility, a reference request is transmitted to each facility. In response to the reference request, the ID of the user at each facility is obtained with the inter-facility user ID associating unit. Also, for a staff ID already authenticated by the staff login authenticating unit, what kind of access right is given in which document category can be identified with the facility-specific document-category-specific access right managing unit of each facility. At the facility, if there is a document category related to the user the staff member has a right to access, the referenced document data is transmitted to the reference request source (an accessible document referencing unit). In other words, the accessible document referencing unit transmits the referenced document data to the staff related to the staff ID already authenticated as reference request source in case where a user ID associated among the facilities exists, and where the document category related to the user that the staff has rights to access at the referenced document data exists. The terminal that has received the referenced document data displays the reference document data, using a multi-facility document display unit.

For ease of explanation, the facility-specific user ID managing unit, the inter-facility user ID associating unit, the facility-specific document category managing unit, the facility-specific document-category-specific recording unit, the facility-specific document-category-specific access right managing unit, and the staff login authenticating unit are mounted in a server in a facility in this example, but some or all of them may be gathered in the central server and be connected by a communication line such as the Internet. In the central server, the respective facility systems may be mounted in the respective physical servers separately from one another, or virtual servers may be formed by logically dividing the same physical server. Further, an actual server may not be formed, and a cloud service may be used in the form of SaaS. Also, the multi-facility document display unit may be mounted in a terminal, or a display image may be created in a server and be transmitted to a terminal screen. Further, the accessible document referencing unit may include part of the reference system described in Conventional Example 2.

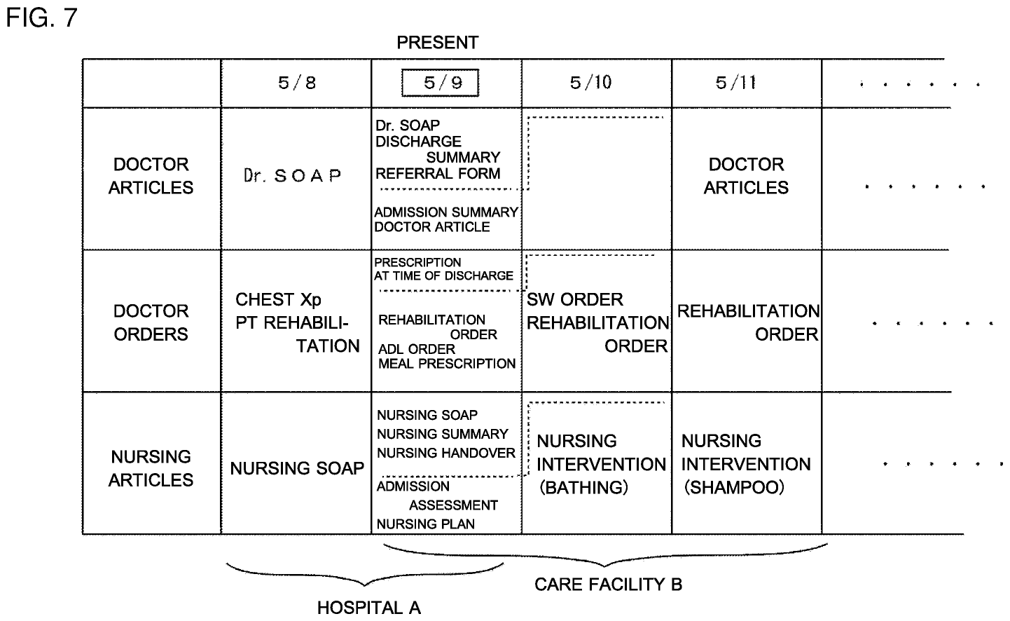

FIG. 7 is an example of a calendar-style screen displayed by the multi-facility document display unit. The abscissa axis indicates dates, and the ordinate axis indicates document category classification frames. In this example, doctor articles, doctor orders, nursing articles, and the like are shown. In this frames in the vertical and horizontal directions, the respective documents are shown in the document classification frames to which the dates on the documents and the document categories belong. The display is as of May 9. The patient was an inpatient at Hospital A until noon, May 9, and has been transferred to Care Facility B in the afternoon on the same day. In the frames for May 10 and later, schedules input with future dates are displayed. With this, articles and a series of instructions can be viewed as a list, and it is easy to recognize situations, even if a patient is transferred between facilities. Thus, an accident due to overlooking can be prevented. The document category classification frames can be appropriately set for each facility and each staff member. In other words, the multi-facility document display unit causes display of a calendar-style screen including a row indicating dates, and a column indicating document category, a cell indicating the multi-facility document, and an object indicating the facility related to the multi-facility document. The multi-facility document display unit causes display of the calendar-style screen including a dividing line between the multi-facility documents within the cell in case where the cell indicates the multi-facility documents related to different facilities. The multi-facility document display unit displays the object proximity of the row, the column or the cell related to the referenced document data.

In this example, a “doctor article” is “Doctor SOAP” at another facility. In this manner, documents with similar contents have slightly different document category names or particulars depending on facilities in many cases. Documents with similar contents provides a better list display when displayed as a series. A document category similarity correspondence table is created among the facilities, and similar document categories are displayed in the same classification frame so that temporal changes in the contents can be easily grasped (an example of an inter-facility similar document category associating unit).

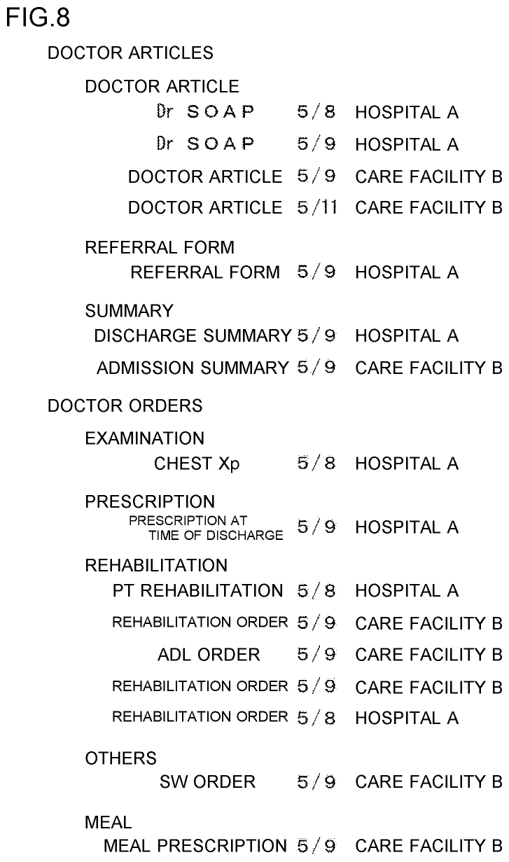

FIG. 8 shows a display of the same document group as that shown in FIG. 7. In FIG. 8, however, only the doctor-related items are displayed in chronological order in the respective document categories (another example of an inter-facility similar document category associating unit). Although the names of the document categories vary with facilities, documents of the other facility are displayed under similar standard facility document categories in a document list of each document category of the standard facility, in accordance with the document category similarity correspondence table. With this, even if document category names and particulars vary with facilities, documents with similar contents are displayed as a series, and an excellent list display can be formed.

The accessible document referencing unit gathers documents of more than one facility. In the displays shown in FIGS. 7 and 8, it is possible to recognize the facility to which a document belongs simply from the date and time, for example. However, at a home care facility, there exist documents created by staff members belonging to different facilities, such as a doctor from a hospital, a home-visiting nurse and a physical therapist from a home-visit nurse station, and a caregiver from a home-visit care station. As a result, it becomes difficult to recognize the facility to which a document belongs. Therefore, when each facility references a document through the accessible document referencing unit, tag information indicating the referencing facility is attached to the referenced document data, so that the origin of the document becomes clear. With this, it becomes easier to display documents of each facility. Further, when there is a document edit request from a staff member, it becomes clear at which facility an inquiry as to whether the staff member has a right to edit the document should be made.

Here, the “tag information indicating the referencing facility” may be “<referencing facility> home-visit nurse station XY</referencing facility>” in a case where the document data is in the XML format, or may be “referencing facility: home-visit nurse station XY” in a case where the document data is in the JSON format. In a relational database format, an item “referencing facility” may be formed. In any case, data indicating the referencing facility should be attached to the referenced document data.

Medical or care facilities manage a staff list in the form of a list of employees or the like at each facility. All the records of the medical and nursing care services provided by staff members, including the information gathering and the planning, are managed at each facility. A patient is transferred from an acute hospital to a recovery hospital to a care facility to a home care service, in accordance with stages of disease. Documents created in relation to medical and nursing care are created, edited, or deleted at the facility to which the patient has been transferred (a principal facility), and should then be saved and managed. The staff members providing medical and nursing care services to the patient have rights to create, edit, or delete documents at the principal facility, and need to create, edit, or delete documents with the rights.

In a case where the facility to which a patent is to be transferred (the next “principal facility”) has already been decided, documents in relation to medical and nursing care of future dates after the transfer should be created at the next principal facility to which the patient is to be transferred, instead of the current principal facility. For this reason, to create, edit, and delete documents at future principal facilities as well as the current principal facility, it is necessary to designate a principal facility, and a right to create, edit, or delete documents at the facility needs to be authenticated through the staff login authenticating unit.

Staff login authentication may not be performed directly on the principal facility, but additional login authentication may be performed on another necessary principal facility after the staff member has logged in at the principal facility. If the same staff ID and the same password are used, the additional login authentication is automatically performed. In such a case, security becomes slightly weaker. Therefore, depending on the adopted operation policy, the same staff member may have a different staff ID and a different password at each principal facility. After staff login authentication is performed at the principal facility, access rights in the respective document categories are given in accordance with FIG. 5.

Every time a document is created, edited, or deleted, a principal facility may be designated, and staff login authentication at the facility may be performed as necessary. However, the process of identifying a principal facility which is currently-used, and further which a patient is transferred to, is often complicated. To avoid this, a table for managing a currently-used principal facilities, and further, if necessary, future principal facilities is created in advance, for each user. With this, it is possible to identify the corresponding principal facility by designating a user ID and a date on which a document is to be created, edited, or deleted. If staff login authentication has not been performed on the corresponding principal facility, additional staff login authentication should be performed so that the document can be smoothly created, edited, or deleted.

Although an embodiment has been described so far, specific structures of the present invention are not limited to the above embodiment, and design changes and the like within the scope of the invention are included in the present invention.

For example, the facility-specific document-category-specific document recording unit of the present invention records document data in the XML format, and the accessible document referencing unit also references document data in the XML format. However, it is possible to adopt JSON, HL-7, CSV, or the like, as long as recording and transmission/reception can be performed. Alternatively, a document recorded in a relational database may be converted into a format suitable for referencing.

In an embodiment, the integrated multi-facility electronic medical record system comprises a plurality of the terminals including a processor connected to a memory, the facility-specific server including a processor connected to a memory, and the shared server including a processor connected to a memory.

The terminal includes the processor as an arithmetic device, the memory as a main storage device, an auxiliary storage device, an input device, an output device, a communication device, and a bus interface for interconnection. The terminal may adopt a hardware configuration of generic computing devices (generic computers).

The facility-specific server includes the processor as an arithmetic device, the memory as a main storage device, an auxiliary storage device, a communication device, and a bus interface for interconnection. The facility-specific server may include an input device and an output device. The facility-specific server may adopt a hardware configuration of generic computing devices (generic computers).

The shared server includes the processor as an arithmetic device, the memory as a main storage device, an auxiliary storage device, a communication device, and a bus interface for interconnection. The shared server may include an input device and an output device. The shared server may adopt a hardware configuration of generic computing devices (generic computers).

The processor included in the terminal plays the action/role of the terminal including examples of the unit/means/step/function included in the terminal. The processor included in the terminal exerts the effect of the unit/means/step/function included in the terminal.

The processor included in the facility-specific server plays/causes the action/role of the facility-specific server including examples of the unit/means/step/function including the facility specific user ID managing unit, the inter-facility user ID associating unit, the facility specific document category managing unit, the facility specific document category specific document recording unit, and the facility specific document category specific access right managing unit.

The processor included in the shared server plays the action/role of the shared server including examples of the unit/means/step/function including the accessible document referencing unit and the multi facility document display unit.

1. An integrated multi-facility electronic medical record system comprising:
a plurality of terminals;
a facility-specific server including a processor connected to a memory that:
manages IDs of facility users at each facility among a plurality of facilities;
manages document categories being used at each facility among the plurality of facilities;
records document data for each document category at each facility among the plurality of facilities;
manages access rights of staff for each document category at each facility among the plurality of facilities;
authenticates staff for each document category at each facility based on a staff ID and a password received from the terminal;
associates different user IDs indicating same user between the facilities,
transmits the referenced document data to the staff related to the staff ID already authenticated as reference request source in case where a user ID associated among the facilities exists, and where the document category related to the user that the staff has rights to access at the referenced document data exists; and
a shared server including a processor connected to a memory that:
references, for each facility among the facilities and each document category, a document the staff has rights to access at the plurality of facilities, the document relating to a designated user;
attaches tags to referenced document data, the tags each indicating reference facility as a reference source;
causes display of a calendar-style screen based on a referenced multi-facility document on the terminal based on the referenced document data with the tags transmitted to the terminal;
causes display of the calendar-style screen including a row indicating dates, a column indicating document category, a cell indicating the multi-facility document, and an object indicating the facility related to the multi-facility document;
causes display of the calendar-style screen including a dividing line between the multi-facility documents within the cell in case where the cell indicates the multi-facility documents related to different facilities; and
causes display of, in similar manners, documents classified under similar document categories at different facilities on the terminal.

2. The integrated multi-facility electronic medical record system according to claim 1, wherein the plurality of terminals each include a processor that
designates a principal facility, and allows document creation, edit, and deletion only in a document category in which the staff have rights to create, edit, and delete a document among document categories at the designated principal facility.

3. The integrated multi-facility electronic medical record system according to claim 2, wherein the processor of the shared server
records, for each user, a facility being currently used, a facility to be used in the future, and a period of use, and, when a user and a date are designated, automatically designates the principal facility.
